SEQUENCE LISTING (36 sequences)

SEQ ID NO: 1 - DNA, 23 bp, Homo sapiens
gttcggttttagggttcggagat

SEQ ID NO: 2 - DNA, 26 bp, Homo sapiens
caaaatataccgacttcttcgattcg

SEQ ID NO: 3 - DNA, 26 bp, Homo sapiens
ttttataggggtagcgattagcgttg

SEQ ID NO: 4 - DNA, 22 bp, Homo sapiens
ctctaaaacgcgctcaccgaaa

SEQ ID NO: 5 - DNA, 22 bp, Homo sapiens
tttgggtcgggttgggtcgttt

SEQ ID NO: 6 - DNA, 26 bp, Homo sapiens
ggatattgtatgcgtttgcgtagatt

SEQ ID NO: 7 - DNA, 28 bp, Homo sapiens
cataacaacaacgtacctctacgtcctc

SEQ ID NO: 8 - DNA, 29 bp, Homo sapiens
tcgttttcgtagttagagaggttcggatg

SEQ ID NO: 9 - DNA, 19 bp, Homo sapiens
cgggcgaagcgtacggtgt

SEQ ID NO: 10 - DNA, 25 bp, Homo sapiens
cgcaaaataactaacccgactacga

SEQ ID NO: 11 - DNA, 24 bp, Homo sapiens
tgcgttttatcgatcgtacgtgtt

SEQ ID NO: 12 - DNA, 20 bp, Homo sapiens
cccgactacgctcctccgac

SEQ ID NO: 13 - DNA, 29 bp, Homo sapiens
gatttagtagtcgttggtataagttgcgt

SEQ ID NO: 14 - DNA, 21 bp, Homo sapiens
cgataaatccgcgcactcgaa

SEQ ID NO: 15 - DNA, 24 bp, Homo sapiens
cggagacgtgggagcgtttttttg

SEQ ID NO: 16 - DNA, 21 bp, Homo sapiens
cgtttcggaatcgacgtgagc

SEQ ID NO: 17 - DNA, 22 bp, Homo sapiens
aaattcgactccgaacgaacga

SEQ ID NO: 18 - DNA, 19 bp, Homo sapiens
tcggttcgtggaggcggtt

SEQ ID NO: 19 - DNA, 24 bp, Homo sapiens
agtgataggttggttcggcgtagt

SEQ ID NO: 20 - DNA, 18 bp, Homo sapiens
ctcgcgctacccccgaaa

SEQ ID NO: 21 - DNA, 21 bp, Homo sapiens
aaccctaccgccgcacccgct

SEQ ID NO: 22 - DNA, 21 bp, Homo sapiens
cggtcggttacgtcgtcgaat

SEQ ID NO: 23 - DNA, 24 bp, Homo sapiens
caacgaaacgaaaacgattacgaa

SEQ ID NO: 24 - DNA, 30 bp, Homo sapiens
cgttatgggttttttttcgtattcgtatgt

SEQ ID NO: 25 - DNA, 21 bp, Homo sapiens
ttgagattggcggaggcggtt

SEQ ID NO: 26 - DNA, 25 bp, Homo sapiens
ccattttcttttaaacgaaactcgc

SEQ ID NO: 27 - DNA, 21 bp, Homo sapiens
tgtgggcggttacgtgttgcg

SEQ ID NO: 28 - DNA, 25 bp, Homo sapiens
ttgtatttgacgggtaatttgagcg

SEQ ID NO: 29 - DNA, 25 bp, Homo sapiens
cttaaaacgtttaaactcccgcaac

SEQ ID NO: 30 - DNA, 30 bp, Homo sapiens
ggagaataatcgttatatcgttatcgacgg

SEQ ID NO: 31 - DNA, 21 bp, Homo sapiens
gggttttagttcggagcgggt

SEQ ID NO: 32 - DNA, 21 bp, Homo sapiens
gaccaaaacgaccccgaacaa

SEQ ID NO: 33 - DNA, 24 bp, Homo sapiens
tttggcgaggaaggtatggtcggt

SEQ ID NO: 34 - DNA, 20 bp, Homo sapiens
ggtgacggttgggggcgtat

SEQ ID NO: 35 - DNA, 22 bp, Homo sapiens
aaaatacgccaaccgcatacga

SEQ ID NO: 36 - DNA, 25 bp, Homo sapiens
ggtagtagatgttgcgggtgtcggt

(19) United States
(12) Patent Application Publication
(10) Pub. No.: US 2022/0251663 A1
(43) Pub. Date: Aug. 11, 2022

(54) DNA METHYLATION BIOMARKERS FOR CANCER DIAGNOSING AND TREATMENT

(71) Applicant: ARIZONA BOARD OF REGENTS ON BEHALF OF THE UNIVERSITY OF ARIZONA, Tucson, AZ (US)

(72) Inventors: Bernard W. Futscher, Tucson, AZ (US); Lukas Vrba, Tucson, AZ (US); Mark A. Nelson, Tucson, AZ (US)

(21) Appl. No.: 17/619,116

(22) PCT Filed: Jun. 5, 2020
(86) PCT No.: PCT/US20/36342

§ 371 Date: Dec. 14, 2021

Related U.S. Application Data
(60) Provisional application No. 62/861,934, filed on Jun. 14, 2019.

(51) Int. Cl. C12Q 1/6886; C12Q 1/6806

(52) U.S. Cl. CPC C12Q 1/6886; C12Q 1/686; C12Q 2600/154; C12Q 1/6806

(57) ABSTRACT

Cancer is the second most common cause of death worldwide, identification of cancer-specific DNA methylation events released by tumors into blood can be used for cost-effective, minimally invasive diagnostics and monitoring of cancer. The present invention clinically tested a set of ten DNA methylation specific qPCR amplicons, designed to detect most common human carcinoma types, in cell free DNA extracted from plasma fraction of blood samples from healthy controls and non-small cell lung cancer (NSCLC) cases. The DNA methylation biomarkers distinguish lung cancer cases from controls with high sensitivity and specificity (AUC=0.956), and furthermore, the signal from the markers depends on the tumor size and decreases after surgical resection of lung tumors. These observations indicate clinical value of these DNA methylation biomarkers for minimally invasive diagnostics and monitoring of NSCLC. It is predicted that these DNA methylation biomarkers will detect additional carcinoma types as well.

### CROSS-REFERENCES TO RELATED APPLICATIONS

This application claims benefit of U.S. Provisional Application No. 62/861,934 filed Jun. 14, 2019, the specification(s) of which is/are incorporated herein in their entirety by reference.

### REFERENCE TO A SEQUENCE LISTING

Applicant asserts that the information recorded in the form of an Annex C/ST.25 text file submitted under Rule 13ter.1(a), entitled UNIA_20_04_PCT_Sequence_ListingST25.txt, is identical to that forming part of the international application as filed. The content of the sequence listing is incorporated herein by reference in its entirety.

### FIELD OF THE INVENTION

The present invention relates to a method of preparing cell free DNA (cfDNA) more particularly to a method of treating/detecting cancer based on cfDNA methylation levels.

### BACKGROUND OF THE INVENTION

Cancer is the second most common cause of death worldwide. Earlier detection of cancer or its recurrence could improve the treatment and management of the disease. Therefore, to allow for more frequent cancer screening, techniques for minimally invasive and cost-effective cancer diagnosis and monitoring are needed.

Blood contains a small amount of cell free DNA (cfDNA) that can be recovered from plasma or serum samples and is mostly fragmented to a single nucleosome size. cfDNA from healthy individuals is comprised mostly of DNA released from dead hematopoetic cells. However, in cancer patients, additional DNA derived from tumor cells is present. When tumor cells die, their DNA is released into a bloodstream termed circulating tumor DNA (ctDNA) and becomes part of cfDNA. The amount of ctDNA in cfDNA varies depending on cancer type and the disease progression. In general, the addition of ctDNA to the blood results in the overall increase of cfDNA which by itself could be indicative of the disease.

Nonetheless, specific identification of ctDNA within cfDNA samples can largely increase the sensitivity and specificity of cancer detection, especially in earlier stages of the disease when the overall increase of cfDNA amount might not be significant; it can also allow for sensitive monitoring of the residual disease after intervention. Tumor DNA differs from normal cell DNA in several aspects that allow specific detection of ctDNA; these include tumor specific mutations, altered DNA copy numbers and DNA methylation. Overall, specific identification of tumor derived ctDNA in cfDNA samples from blood or other liquid biopsies can be used for minimally invasive diagnosis, appropriate treatment, and monitoring of cancer.

### BRIEF SUMMARY OF THE INVENTION

The fundamental differences between DNA from normal and tumor cells could be found in the epigenome represented by tumor specific changes in DNA methylation. DNA methylation is an optional covalent epigenetic modification of cytosine residues in the CpG sequence context. There are about 28 million CpGs in the human genome. These CpGs are distributed non-randomly and a large fraction of CpGs is located in CpG rich regions called CpG islands. CpG islands are located predominantly at gene promoters and other regulatory regions. In normal cells most of the CpGs are methylated with the exception of CpG islands. Tumor cells have altered epigenome with global DNA hypomethylation and promoter and CpG island specific DNA hypermethylation.

Cell type specific DNA methylation patterns help to determine and keep cellular identity of normal cells while tumor cells have profoundly altered epigenome with two kinds of changes in DNA methylation. First, the cancer cells improperly co-opt some of the DNA methylation changes found in different normal cell types e.g., the presence of mesenchymal cell type specific DNA methylation in carcinomas may be indicative of EMT21, however this is not suitable as a cancer specific marker since it is present also in normal mesenchymal cells and therefore will be present in cfDNA of healthy donors and would result in false positive diagnosis.

Second, cancer cells contain many aberrant DNA methylation changes that do not occur in any normal cells, and these DNA methylation changes are therefore suitable for specific detection of ctDNA in cfDNA samples from plasma or other liquid biopsies. DNA methylation specific qPCR is sensitive enough to detect the presence of even few methylated copies of ctDNA in a typical cfDNA sample. In addition, qPCR is relatively quick and inexpensive. Since tumors have aberrantly methylated many DNA regions, the detection of tumor specific DNA methylation could be performed in multiple genomic loci; this increases the sensitivity of the technique. In summary, the detection of tumor specific DNA methylation in cfDNA from liquid biopsies could be used for diagnosis, appropriate treatment, and monitoring of cancer; the technique would be sensitive, relatively quick and cost effective while minimally invasive.

It is an objective of the present invention to provide a method that allow for the preparation of cell free DNA (cfDNA) more particularly to a method of treating/detecting cancer based on cfDNA methylation levels, as specified in the independent claims. Embodiments of the invention are given in the dependent claims. Embodiments of the present invention can be freely combined with each other if they are not mutually exclusive.

The present invention features a method of preparing a deoxyribonucleic acid (DNA) fraction from a subject useful for analyzing genetic loci involved in DNA methylation. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two biomarkers wherein the DNA fraction comprises a plurality of genetic loci of the cfDNA. In some embodiment, the cfDNA is quantified and analyzed for methylation as a plurality of genetic loci.

The present invention may also feature a method of treating a plurality of cancers by administrating anti-cancer therapeutics in a subject with cancer. In some embodiment, the method comprises determining a subject's DNA methylation level. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two biomarkers wherein the DNA fraction comprises a plurality of genetic loci of the cfDNA. In some embodiment, the cfDNA is quantified and analyzed for methylation as a plurality of genetic loci.

The present invention may also feature a method of detecting one or more cancers from a plurality of different cancer types in a subject. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two biomarkers wherein the DNA fraction comprises a plurality of genetic loci of the cfDNA. In some embodiment, the cfDNA is quantified and analyzed for methylation as a plurality of genetic loci.

One of the unique and inventive technical features of the present invention is a method of preparing methylated cfDNA to detect and treat a plurality of cancers. Without wishing to limit the invention to any theory or mechanism, it is believed that the technical feature of the present invention advantageously provides for a method that is minimally invasive and a cost effective procedure that allows for detection of a plurality of cancer types using a set of cfDNA methylation biomarkers. The present invention allows for timely results, within two days of the blood collection, in a clinical setting. Prior references have used methods that analysis whole cfDNA methylomes, however this approach can be costly and time consuming making it irrelevant in a clinical setting. None of the presently known prior references or work has the unique inventive technical feature of the present invention. Furthermore, the prior references teaches away from the present invention. For example, other methods of detecting cancer using cfDNA methylation use single or multiple markers to detect a single cancer type. Furthermore, the inventive technical features of the present invention contributed to a surprising result that this approach can distinguish the presence of pancreatic cancer from benign cyst and healthy volunteer in cDNA (FIG. 1). Furthermore, a set of these DNA methylation biomarkers can predict which pre-invasive lung carcinoma in situ lesion are precursors to squamous cell carcinoma (FIG. 2).

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows that the approach of the present invention can distinguish the presence of pancreatic cancer from benign cyst and healthy volunteer.

FIG. 2 shows that the present invention can predict which pre-invasive lung carcinoma in situ lesions are precursors to squamous cell carcinoma.

FIG. 3A shows a flowchart of a study disclosed herein

FIG. 3B shows a human ideogram showing chromosomal locations of DNA methylation biomarkers.

FIG. 4 shows the validation of the DNA methylation biomarker set on independent cancer sample cohorts from the GEO. Normal whole blood cohort (GSE72773) and respective normal tissues (NT) were used as controls. The plots show DNA methylation of the marker set in individual tumor samples in comparison to normal blood samples and respective normal tissue (NT) samples. The DNA methylation data from the normal blood cohort are shown only in the first panel and are represented in the additional panels by the horizontal dashed lines showing the 95^{th} percentile of the cumulative DNA methylation of the normal blood cohort. The horizontal dotted lines indicate the 95^{th} percentiles of the cumulative DNA methylation of the respective NT cohorts. The AUCs were calculated using the respective tumor cohort and the normal blood cohort or respective NT as a normal reference for each cancer cohort.

FIGS. 5A-5B show the DNA methylation biomarker set differentiates between lung cancer cases and healthy controls with high sensitivity and specificity. FIG. 5A shows mean DNA methylation signal per marker of the full 10 marker set (see Table 4) for the control group of 47 healthy volunteers and for the group of 18 NSCLC cases. P-value shown is for Wilcoxon rank sum test. FIG. 5B shows the receiver operating characteristic (ROC) analysis of the marker set signal from 47 controls and 18 NSCLC cases. AUC—area under the curve, CI—confidence interval.

FIGS. 6A-6D show the effect of age on DNA methylation biomarker performance and improved performance of the five biomarker subset. FIG. 6A shows the age distribution of the entire control cohort, control cohort split into three sub-cohorts by age and NSCLC patient cohort. FIG. 6B shows the ROC analysis of the performance of the full 10 marker set using only the oldest third of healthy volunteers as control. FIG. 6C shows the ROC analysis of the performance of the five marker subset using only the oldest third of healthy volunteers as control. FIG. 6D shows the ROC analysis of the performance of the five marker subset using all healthy volunteers as control.

FIGS. 7A-7D show the DNA methylation biomarker signal depends on tumor size and disease stage and decreased after tumor removal. Correlation between the DNA methylation marker signal and tumor size (FIG. 7A) and disease stage (FIG. 7B). DNA marker methylation in pairs of blood samples collected before surgical resection of tumor, and three days (FIG. 7C) or three months (FIG. 7D) after the tumor resection. Y axis shows mean DNA methylation signal per marker of the full ten marker set

FIG. 8 shows a schema of the two-step qPCR. First step: all methylated template molecules extracted from 2 ml of plasma are in contact with all primer pairs and therefore amplified. Second step: since all the available template was pre-amplified in the first step there is enough copies of each methylated marker to be representatively divided into individual marker specific reactions for quantification and therefore could be successfully detected even if the original amount was only several molecules.

FIG. 9 shows DNA methylation signal from the whole 10 marker set on a cohort of 47 healthy subjects (left part) and 18 non-small cell lung cancer patients (right part). The 95^{th} percentile of the cumulative DNA methylation of the control cohort is represented by the horizontal dashed line.

FIG. 10 shows the performance of individual markers. ROC analysis of signal from individual markers using 18 lung cancer patients and 47 healthy subjects as control.

FIG. 11 shows the analysis of DNA methylation signal of individual markers between sexes of healthy subjects. The first ten panels show data for individual markers. The last two panels show combined signal from all 10 markers and age, respectively.

FIG. 12 shows the relation between the DNA methylation of individual markers and the age of healthy subjects. The last panel shows the relation between the signal from the whole marker set and age. The brown lines indicate the linear model fit. The Spearman correlation coefficients rho and the corresponding p-values are listed above each plot.

FIG. 13 shows a depiction of a DNA methylation amplicon region example.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

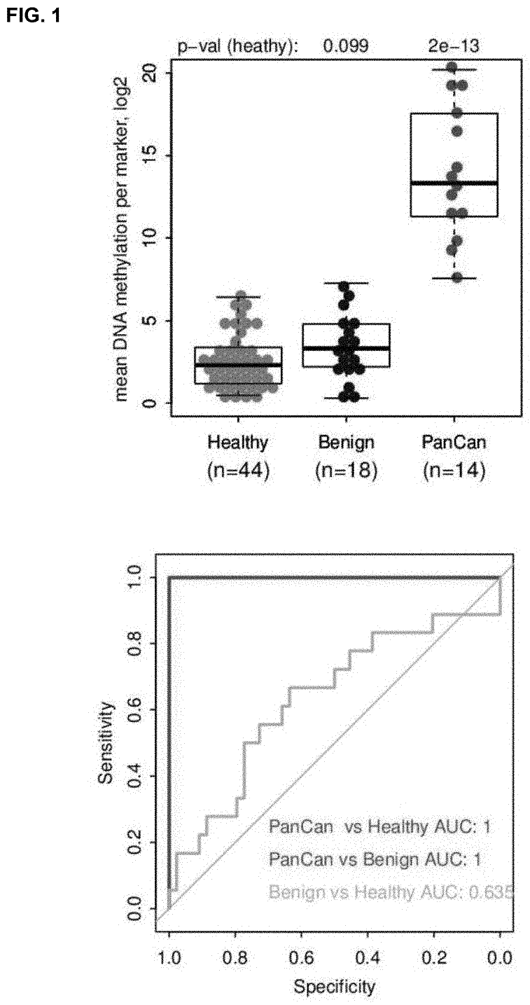

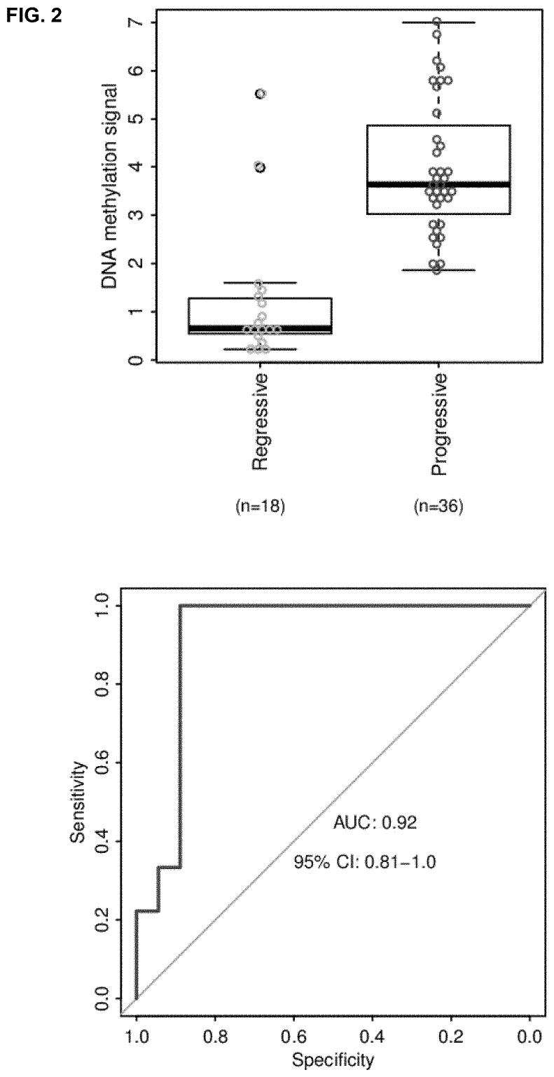

Referring now to FIGS. 1-13, the present invention features a method of preparing methylated cfDNA to detect and treat a plurality of cancers.

The present invention features a method of preparing a deoxyribonucleic acid (DNA) fraction from a subject useful for analyzing genetic loci involved in DNA methylation. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two methylation biomarkers wherein the DNA fraction comprises a plurality of genetic loci of the cfDNA. In some embodiment, the cfDNA is quantified and analyzed for methylation as a plurality of genetic loci.

As used herein “deoxyribonucleic acid (DNA) methylation” refers to an optional epigenetic modification of a cysteine residue in the sequence context CpG. As used herein “CpG or CG sites” refer to regions of DNA where a cytosine nucleotide is followed by a guanine nucleotide in the linear sequence of bases along its 5′→3′ direction.

In some embodiment the DNA is extracted from a substantially cell-free sample is of blood plasma or blood serum. As used herein “cell free DNA (cfDNA)” may refer to all non-encapsulated DNA in the blood. In some embodiment, cfDNA are nucleic acid fragments may enter the blood stream during apoptosis or necrosis. In some embodiment cfDNA may contain circulating tumor DNA (ctDNA). As used herein “ctDNA” may refer to DNA that comes from cancerous cells or tumors in the bloodstream that is not associated with cells.

In some embodiment, the cfDNA is treated with sodium bisulfate (BS). As used herein “sodium bisulfite treatment” may refer to a reaction that protects methylated cytosines from conversion, whereas unmethylated cytosines are converted into uracil. In some embodiment, after PCR the converted uracils are recognized as thymines, whereas the methylated cytosines will appear as cytosines.

In some embodiment methylated cfDNA is amplified by use of a polymerase chain reaction (PCR). As used herein “PCR” may refer to a method to rapidly make multiple copies of specific DNA samples from a mixture of DNA molecules. In another embodiment, the methylated cfDNA is quantified and analyzed by quantitative PCR (qPCR). As used herein “qPCR” may refer to a method to determine absolute or relative quantities of a known sequence in a sample. In some embodiment the quantified sequence is analyzed to determine the methylation levels of the cfDNA in the sample.

In some embodiment, the methylated biomarkers are selected from a group consisting of those with a genomic position of: chr11:43602597-43603195, chr2:105458914-10545960, chr1:169369385-16939694, chr16:23847075-23847811, chr2:162283352-162283956; chr19:38182805-38183407, chr5:16179798-16180395, chr7:49812797-49813366, chr5:528326-528904, and chr7:27196014-27196581.

In some embodiment 1 to 15 markers are selected for amplifying methylated cfDNA. In some embodiment 1 to 10 markers are selected for amplifying methylated cfDNA. In some embodiment 2 to 14 markers are selected for amplifying methylated cfDNA. In some embodiment 2 to 12 markers are selected for amplifying methylated cfDNA. In some embodiment 2 to 10 markers are selected for amplifying methylated cfDNA. In some embodiment 2 to 8 markers are selected for amplifying methylated cfDNA. In some embodiment 2 to 6 markers are selected for amplifying methylated cfDNA. In some embodiment 2 to 4 markers are selected for amplifying methylated cfDNA. In some embodiment 2 to 5 markers are selected for amplifying methylated cfDNA. In some embodiment 2 to 6 markers are selected for amplifying methylated cfDNA. In some embodiment 5 to 10 markers are selected for amplifying methylated cfDNA. In some embodiment 8 to 10 markers are selected for amplifying methylated cfDNA.

In some embodiment at least two methylation biomarkers are selected for amplifying methylated cDNA, in some embodiment at least three methylation biomarkers are selected for amplifying methylated cfDNA. In some embodiment at least four methylation biomarkers are selected for amplifying methylated cfDNA. In some embodiment at least five methylation biomarkers are selected for amplifying methylated cfDNA. In some embodiment at least six methylation biomarkers are selected for amplifying methylated cfDNA. In some embodiment at least seven methylation biomarkers are selected for amplifying methylated cfDNA. In some embodiment at least eight methylation biomarkers are selected for amplifying methylated CDNA. In some embodiment at least nine methylation biomarkers are selected for amplifying methylated cfDNA. In some embodiment at least ten methylation biomarkers are selected for amplifying methylated cfDNA.

The present invention may also feature a method of treating a plurality of cancers by administrating anti-cancer therapeutics in a subject with cancer. In some embodiment, the method comprises determining a subject's DNA methylation level. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two biomarkers wherein the DNA fraction comprises a plurality of genetic loci of the cfDNA. In some embodiment, the cfDNA is quantified and analyzed for methylation as a plurality of genetic loci.

In some embodiment the said plurality of different cancer types comprises, urothelial bladder carcinoma (BLCA), breast invasive carcinoma (BRCA), colon adenocarcinoma (COAD), esophageal carcinoma (ESCA), head-neck squamous cell carcinoma (HNSC), lung adenocarcinoma (LUAD), lung squamous cell carcinoma (LUSC), pancreatic adenocarcinoma (PAAD), prostate adenocarcinoma (PRAD), and rectum adenocarcinoma (READ).

In some embodiment the anti-cancer therapeutics consist of one or more of surgery, chemotherapy, radiation therapy, hormonal therapy, targeted therapy (including immunotherapy such as monoclonal antibody therapy) and synthetic lethality.

In some embodiment, a subject's DNA methylation level refers to the amount of methylation found in a subjects cfDNA quantified by qPCR.

The present invention may also feature a method of detecting one or more cancers from a plurality of different cancer types in a subject. In some embodiment, the method comprises extracting DNA for a substantially cell-free sample of blood plasma or blood serum of a subject to obtain cell free DNA (cfDNA). In some embodiment a fraction of DNA is produced by treating the cfDNA with sodium bisulfite (BS) to produce either a set of uracil modified cfDNA and a set of methylated cfDNA and then selectively amplifying only methylated cfDNA with at least two biomarkers wherein the DNA fraction comprises a plurality of genetic loci of the cfDNA. In some embodiment, the cfDNA is quantified and analyzed for methylation as a plurality of genetic loci.

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects.

Accordingly, in some embodiments, biomarker regions include both the position of an example CpG from discovery data as well as a qPCR amplicon region that is 250 bp in both directions. As a result, region sizes typically will be in a range about 550-750 bp

Furthermore, in some embodiment methods herein involve analyzing data from a processed sample to arrive at a degree of confidence based on the level of each DNA methylation biomarker of the panel of DNA methylation markers; and determining a cutoff value; wherein when the degree of confidence is higher than the cutoff value, a diagnosis of cancer. In some embodiment, the methods herein involve monitoring cancer treatment or recurrence, as well as methods of treating cancer based on detecting a type of cancer through methylation biomarkers and then treating the type of cancer detected, are disclosed.

The biomarkers and methods disclosed herein can also be used to monitor or detect cancer recurrence, as well as for the monitoring of treatment effectiveness. Thus, for example, the 5 methylation marker set can be used to detect cell free DNA methylation, whereby a decrease or disappearance of detection indicates treatment effectiveness. Conversely, recurrence of a cancer type is indicated if methylation markers for cancer are detected anew.

As described herein, sensitivity of a biomarker is defined as a biomarker's ability to detect a disease in patients in whom the disease is truly present (i.e., a true positive), and specificity is the ability to rule out the disease in patients in whom the disease is truly absent (i.e., a true negative).

### Example

The following is a non-limiting example of the present invention. It is to be understood that said example is not intended to limit the present invention in any way. Equivalents or substitutes are within the scope of the present invention.

Example 1 describes how an optimal set of DNA methylation markers can be used in a clinical setting to determine if a patient has cancer.

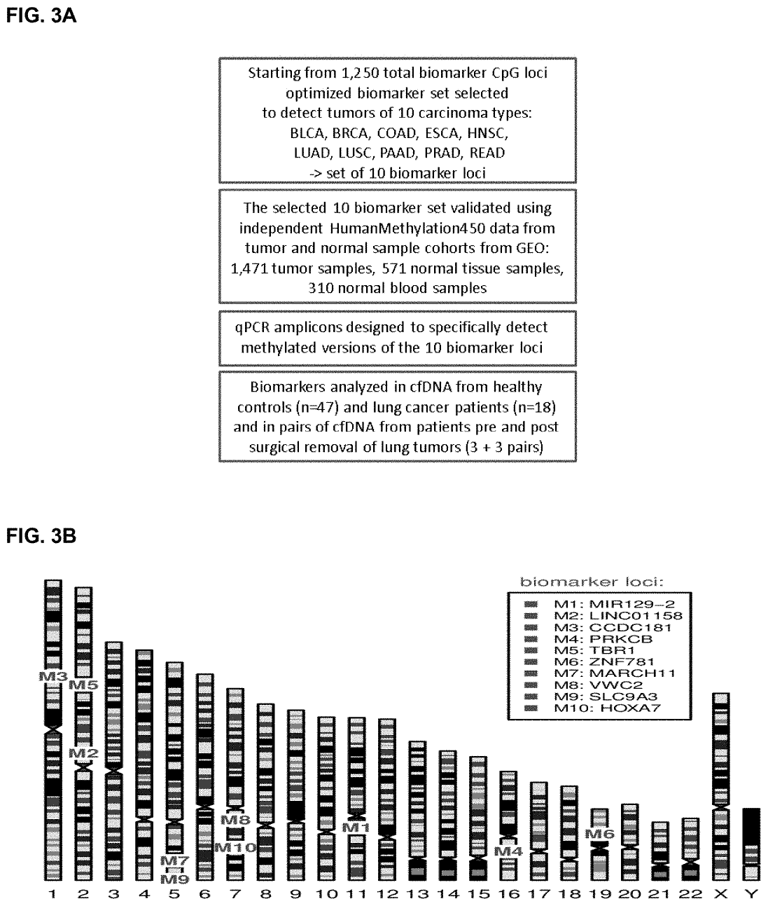

An optimal set of 10 DNA methylation biomarkers that can identify non-small cell lung cancer (NSCLC) (represented by TCGA cancer types LUAD and LUSC) and additional 8 TCGA cancer types (BLCA, BRCA, COAD, ESCA, HNSC, PAAD, PRAD, READ, Table 1A, FIG. 3A) were selected for the study. This optimal set consists of 10 marker loci (Table 1B. FIG. 3B) were tested using independent data from the Gene Expression Omnibus (GEO) database.

Table 1A (below) list the 10 The Cancer Genome Atlas (TCGA) cancer types for which the marker set was designed including GEO cancer cohort names that were used for validation.

| TCGA Cancer Type | | GEO |
|---|---|---|
| Abbreviation | TCGA Cancer Type Name | representative |
| BLCA | Bladder Urothelial Carcinoma | Bladder cancer |
| BRCA | Breast invasive carcinoma | Breast cancer |
| COAD | Colon adenocarcinoma | Colorectal cancer |
| ESCA | Esophageal carcinoma | Esophageal cancer |
| HNSC | Head and Neck squamous | Oral cancer |
| | cell carcinoma | |
| LUAD | Lung adenocarcinoma | NSCLC |
| LUSC | Lung squamous cell | NSCLC |
| | carcinoma | |
| PAAD | Pancreatic adenocarcinoma | Pancreatic cancer |
| PRAD | Prostate adenocarcinoma | Prostate cancer |
| READ | Rectum adenocarcinoma | Colorectal cancer |

Table 1B (below) lists the 10 DNA methylation biomarkers. CpG.ID is specific identification of CpG from Illumina HumanMethylation450 microarray platform, CpG position indicates the physical address of CpG in human genome assembly hg19, and annotation indicates overlapping or nearby located gene

| | | Patent. Region | CpG. ID position | Amplicon genomic |
|---|---|---|---|---|
| Biomarkers | CpG.ID | (hg19) | (hg19) | position (hg19) |
| MIR129-2 | cg14416371 | chr11: 43602597- | chr11: 43602847- | chr11: 43602876- |
| (amplicon 70bp) | | 43603195 | 43602848 | 43602945 |
| LINC01158 | cg08189989 | chr2: 105458914- | chr2: 105459164- | chr2: 105459225- |
| (amplicon 86bp) | | 105459560 | 105459165 | 105459310 |
| CCDC181 | cg00100121 | chr1: 169396385- | chr1: 169396635- | chr1: 169396658- |
| (amplicon 87bp) | | 169396994 | 169396636 | 169396744 |
| PRKCB | cg03306374 | chr16: 23847075- | chr16: 23847325- | chr16: 23847491- |
| (amplicon 71bp) | | 23847811 | 23847326 | 23847561 |
| TBR1 | cg01419831 | chr2: 162283352- | chr2: 162283705- | chr2: 162283602- |
| (amplicon 73bp) | | 162283956 | 162283706 | 162283674 |
| ZNF781 | cg25875213 | chr19: 38182805- | chr19: 38183055- | chr19: 38183080- |
| (amplicon 78bp | | 38183407 | 38183056 | 38183157 |
| MARCH11 | cg00339556 | chr5: 16179798- | chr5: 16180048- | chr5: 16180057- |
| (amplicon 89bp) | | 16180395 | 16180049 | 16180145 |
| VWC2 | cg01893212 | chr7: 49812797- | chr7: 49813088- | chr7: 49813047- |
| (amplicon 70bp) | | 49813366 | 49813089 | 49813116 |
| SLCSA3 | cg14732324 | chr5: 528326- | chr5: 528621- | chr5: 528576- |
| (amplicon 79bp) | | 528904 | 528622 | 528654 |
| HOXA7 | cg07302069 | chr7: 27196014- | chr7: 27196286- | chr7: 27196264- |
| (amplicon 68bp) | | 27196581 | 27196287 | 27196331 |

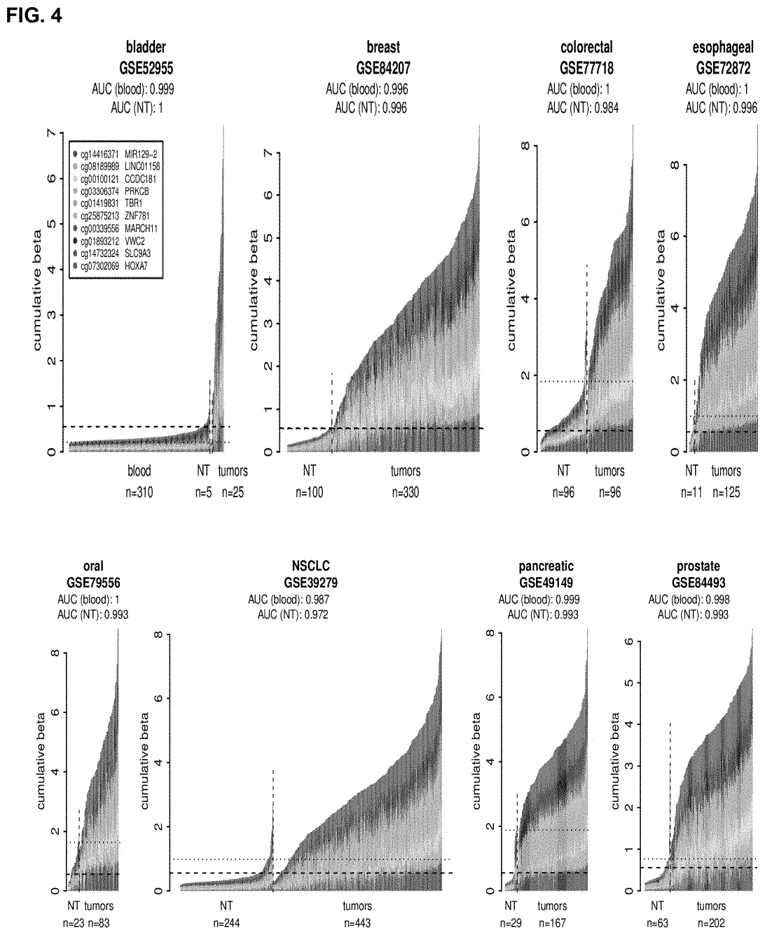

Eight GEO cancer sample cohorts (total n=1,471) representing the 10 TCGA cancer types (Table 1A) were tested against normal blood GEO samples (n=310) as well as respective normal tissue (NT) GEO samples (total n=571) (FIG. 4). The results confirmed that this set of 10 markers can identify, with high sensitivity and specificity (blood reference: AUC 0.987-1.0; respective normal tissue reference: AUC 0.972-1.0), all cancers it was designed for (FIG. 4). These findings show that the selected marker set can differentiate very well tumor specific DNA from DNA originating from normal blood or normal tissue samples. In summary, the optimal biomarker set was able to detect DNA methylation in lung cancer and additional common carcinomas. In addition, these markers can distinguish tumor derived DNA from DNA originating from normal cells.

Ten qPCR amplicons specific for the marker loci and three control amplicons were designed. The marker amplicons were selected to overlap or be as close as possible to the marker CpGs determined by the Illumina HumanMethylation450 microarray (Table 1B). In addition to ten marker amplicons three qPCR amplicons specific for universally methylated loci that serve as cfDNA load controls were designed (Table 2A). The pairs of primers and the probes for all qPCR amplicons were designed to be specific for the methylated sodium bisulfite treated DNA. The size of the amplicons was designed to be as short as possible (60-90 bp) to perform well on the fragmented cfDNA template (Table 2A). Primers and probes were designed to overlap at least 7 CpGs combined (at least two CpGs each, closer to the 3′ end for primers) to be specific only for the methylated template. Where possible, probes from the Human Universal Probe Library Set (Roche Diagnostics, Indianapolis, Ind., USA) were utilized, otherwise custom probes with 5′ 6-FAM—6-carboxyfluorescein and 3′ Iowa Black® FO labels were designed. The primers and the custom probes were manufactured by Integrated DNA Technologies (Coralville, Iowa, USA).

Quantitative PCR specific: to methylated marker regions was chosen in order to detect very small amounts of methylated ctDNA found in cfDNA samples. Ten qPCR amplicons specific for 10 marker loci were designed (FIG. 3B). The qPCR amplicons were invented to overlap the marker CpGs from Table 1B. The primers and probes were designed to be specific for bisulfite converted DNA and to amplify and detect the marker region only when it is methylated as is the case of tumor specific DNA. The size of the amplicons was selected to be as short as possible (Table 2A) to perform well on the fragmented templates like cfDNA.

Table 2A (below) shows the descriptions of the analytical amplicons including the amplicon size and primer and probe sequences.

| | Amplicon | | | | |
|---|---|---|---|---|---|
| | length | | | | |
| Biomarkers | (bps) | Chromosome | Forward Primer | Reverse Primer | Probe Sequence |
| MIR129-2 | 70 | 11 | GTTCGGTTTTAGG | CAAAATATACCGAC | Roche UPL70 |
| | | | GTTCGGAGAT | TTCTTCGATTCG | |
| LINC01158 | 86 | 2 | TTTTATAGGGGTA | CTCTAAAACGCGCT | TTTGGGTCGGGTTG |
| | | | GCGATTAGCGTTG | CACCGAAA | GGTCGTTT |
| CCDC181 | 87 | 1 | GGATATTGTATGC | CATAACAACAACGT | TCGTTTTCGTAGTTA |
| | | | GTTTGCGTAGATT | ACCTCTACGTCCTC | GAGAGGTTCGGATG |
| PRKCB | 71 | 16 | CGGGCGAAGCGT | CGCAMATAACTAA | Roche UPL70 |
| | | | ACGGTGT | CCCGACTACGA | |
| TBR1 | 73 | 2 | TGCGTTTTATCGA | CCCGACTACGCTCC | Roche UPL70 |
| | | | TCGTACGTGTT | TCCGAC | |
| ZNF781 | 78 | 19 | GATTTAGTAGTCG | CGATAAATCCGCGC | CGGAGACGTGGGA |
| | | | TTGGTATAAGTTG | ACTCGAA | GCGTTTTTTTG |
| | | | CGT | | |
| MARCH11 | 89 | 5 | CGTTTCGGAATCG | AAATTCGACTCCGA | TCGGTTCGTGGAGG |
| | | | ACGTGAGC | ACGAACGA | CGGTT |
| VWC2 | 70 | 7 | AGTGATAGGTTGG | CTCGCGCTACCCCC | AACCCTACCGCCGC |
| | | | TTCGGCGTAGT | GAAA | ACCCGCT |
| SLCA3 | 79 | 5 | CGGTCGGTTACGT | CAACGAAACGAAAA | CGTTATGGGTTTTTT |
| | | | CGTCGAAT | CGATTACGAA | TTCGTATTCGTATGT |
| HOXA7 | 68 | 7 | TTGAGATTGGCGG | CCATTTTCTTTTAAA | TGTGGGCGGTTACG |
| | | | AGGCGGTT | CGAAACTCGC | TGTTGCG |
| Controls | | | | | |
| LRRC8A | 81 | 9 | TTGTATTTGACGG | CTTAAAACGTTTAAA | GGAGAATAATCGTT |
| | | | GTAATTTGAGCG | CTCCCGCAAC | ATATCGTTATCGAC |
| | | | | | GG |
| NCOR2 | 74 | 12 | GGGTTTTAGTTCG | GACCAAAACGACCC | TTTGGCGAGGAAGG |
| | | | GAGCGGGT | CGAACAA | TATGGTCGGT |
| TRAP1 | 68 | 16 | GGTGACGGTTGG | AAAATACGCCAACC | GGTAGTAGATGTTG |
| | | | GGGCGTAT | GCATACGA | CGGGTGTCGGT |

Table 2B shows the forward and reverse primer and probe SEQ. ID. NO. by Marker:

| Marker | Forward Primer | Reverse Primer | Probe Sequence |
|---|---|---|---|
| MIR129-2 | SEQ ID NO. 1 | SEQ ID NO. 2 | SEQ ID NO. NA |
| LINC01158 | SEQ ID NO. 3 | SEQ ID NO. 4 | SEQ ID NO. 5 |
| CCDC181 | SEQ ID NO. 6 | SEQ ID NO. 7 | SEQ ID NO. 8 |
| PRKCB | SEQ ID NO. 9 | SEQ ID NO. 10 | SEQ ID NO. NA |
| TBR1 | SEQ ID NO. 11 | SEQ ID NO. 12 | SEQ ID NO. NA |
| ZNF781 | SEQ ID NO. 13 | SEQ ID NO. 14 | SEQ ID NO. 15 |
| MARCH11 | SEQ ID NO. 16 | SEQ ID NO. 17 | SEQ ID NO. 18 |
| VWC2 | SEQ ID NO. 19 | SEQ ID NO. 20 | SEQ ID NO. 21 |
| SLC9A3 | SEQ ID NO. 22 | SEQ ID NO. 23 | SEQ ID NO. 24 |
| HOXA7 | SEQ ID NO. 25 | SEQ ID NO. 26 | SEQ ID NO. 27 |
| LRRC8A | SEQ ID NO. 28 | SEQ ID NO. 29 | SEQ ID NO. 30 |
| NCOR2 | SEQ ID NO. 31 | SEQ ID NO. 32 | SEQ ID NO. 33 |
| TRAP1 | SEQ ID NO. 34 | SEQ ID NO. 35 | SEQ ID NO. 36 |

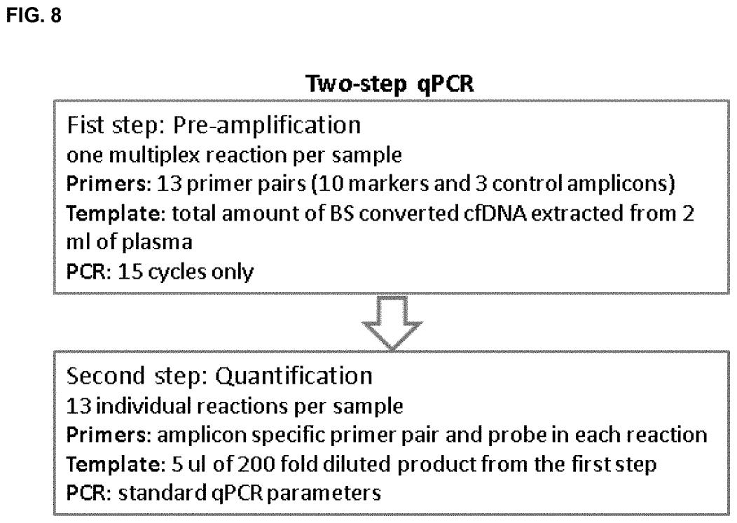

To reduce stochastic effects of low numbers linked to low amounts of methylated ctDNA templates in cfDNA samples a two-step qPCR reaction was adopted as the analytical strategy. In the first step the methylated DNA template is pre-amplified in a multiplex reaction using cocktail of all primers. The product from the first step is then diluted and used in individual standard qPCR reactions to quantify individual markers (FIG. 8). This two-step process allows for ctDNA templates present only in a few copies to be detected since all the templates are equally pro-amplified before the samples are divided into individual amplicon-specific reactions for quantification. In summary, this analytical strategy allows for the detection of DNA methylation of marker loci in plasma cfDNA samples.

Using the above described approach, the cfDNA from healthy donors and lung cancer patients was analyzed. The cfDNA was extracted from plasma samples of 47 healthy volunteers and 18 NSCLC patients (Table 3) recruited between 2018 and 2019 at the University of Arizona, Tucson, Ariz., USA. Institutional Review Board Approval No 1803355376 was obtained prior to the study initiation and all patients and healthy volunteers signed the informed consent. The cancer cohort consisted of stage I-III NSCLC patients (Table 3), here the blood draws were performed before surgical resection of tumors and some of these patients had follow up draws either 3 days or 3 months after the surgery. In addition, cancer cohort contained several stage IV (metastatic) NSCLC patients (Table 3) that were undergoing various forms of treatment. All cases had pathologically confirmed non-small cell lung cancer at the time of blood draw.

Table 3 (below) shows the basic clinical characteristics of lung cancer patients (cases) and healthy volunteers (controls) whose plasma was used in the study.

| | Characteristics: | | | | | | | | | |
|---|---|---|---|---|---|---|---|---|---|---|
| | Age (years): | | Sex: | | Tumor Type: | | Disease Stage: | | | |
| | Range | Median | Male | Female | LUAD | LUSC | I | II | III | IV |
| Cases | 33-82 | 70 | 6 | 12 | 15 | 3 | 5 | 3 | 2 | 8 |
| (n = 18) | | | (33%) | (67%) | (83%) | (17%) | (28%) | (17%) | (11%) | (44%) |
| Controls | 18-85 | 48 | 16 | 31 | — | — | — | — | — | — |
| (n = 47) | | | (34%) | (66%) | | | | | | |

Whole blood was collected in Streck cell-free DNA BCT tubes (La Vista, Nebr.), and stored for no longer than 3 days at room temperature until processing. Collection of plasma was done by spinning the BCT tubes at 1,600 g for 10 min at RT, the plasma fraction was then transferred to 2 ml microfuge tubes. The plasma was then spun at 16,000 g for 10 min at room temperature to remove residual cellular debris. The plasma was then carefully transferred to a new 2 ml microfuge tube and stored at −80° C. cfDNA was extracted from 2 ml of plasma using Qiagen QIAamp Circulating Nucleic Acid Kit according to the manufacturer's instructions, eluted in 50 μl into low bind tubes (1.7 ml Microtube (Maximum Recovery) Cat #22-281LR, Olympus Plastics, Genesee Scientific, El Cajon, Calif.) and stored at −80° C.

The whole amount of cfDNA from 2 ml of plasma was sodium bisulfite (BS) treated using EZ DNA Methylation-Gold Kit (Zymo Research, Irvine, Calif., USA) according to the manufacturer's instructions and eluted in 20 μl of water into low bind tubes. First round PCR amplification was performed in a 50 μl reaction volume using 25 μl of PerfeCta qPCR SuperMix Low ROX (Quanta Biosciences. Gaithersburg, Md., USA), 5 μl of 10× mix of all amplicon primers (final concentration 385 nM each primer) and 20 μl of BS converted cfDNA. The reaction conditions were denaturation at 95° C. for 3 min, and then 15 cycles of 95° C. for 15 s, 57° C. for 30 s, and 72° C. for 30 s. The reaction product was then diluted 200 fold and used in the second step—qPCR. The qPCR mixture consisted of 10 μl of PerfeCta qPCR SuperMix Low ROX, 500 nM each amplicon specific primer, 200 nM amplicon specific probe and 5 μl of the 200 fold diluted product from the first step in 20 μl total reaction volume. The qPCR was conducted on ABI Prism 7500 Sequence Detection System (Applied Biosystems. Foster City, Calif., USA), the reaction conditions were 95° C. denaturation for 3 minutes followed by 50 cycles of 95° C. for 15 seconds and 60° C. for 45 seconds.

The threshold cycles (Cts) for individual markers were determined using fixed marker specific thresholds to keep consistency between individual qPCR runs. Although the qPCR was run for 50 cycles the data generated after 40 cycles were not adding additional resolution between the groups and therefore undetermined Cts or Cts higher than 40 were set to 40. The data were then converted by a formula 40−Ct. This way Ct 40 was set as a background (zero) and the values that are still in log 2 transformed scale but are increasing with the level of DNA methylation specific signal were obtained. These minimally processed values for all markers or the means of these values for all markers or marker subsets were used in the plots and ROC analysis. Since the DNA methylation signal from markers spans several orders of magnitude, nonparametric tests were used to test differences between groups (Wilcoxon rank sum test) or correlation between variables (Spearman's rank correlation coefficient). The optimal marker subset was determined by running ROC analysis for all possible 1023 marker combinations and selecting a marker subset with the largest AUC. Where indicated, the marker methylation data were normalized for cfDNA load using the mean signal from the three universally methylated control amplicons from Table 2A.

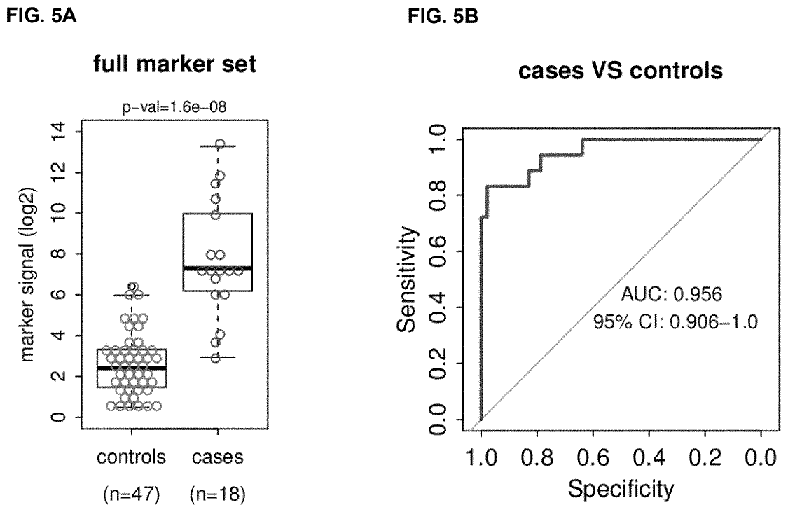

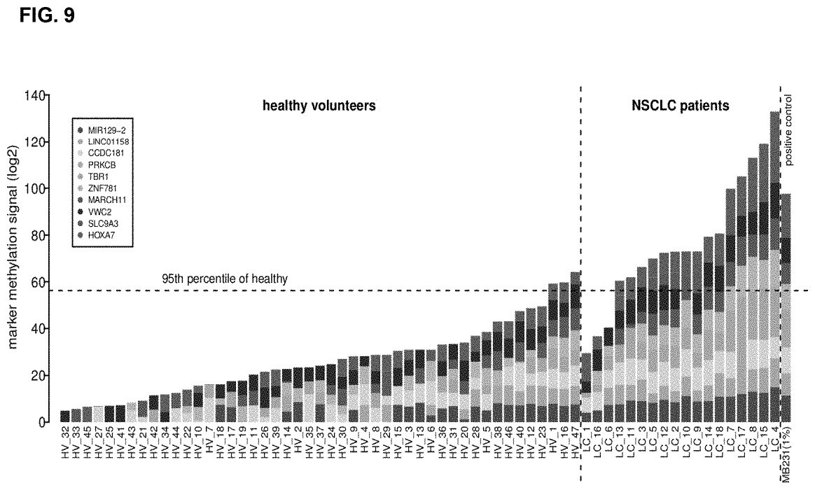

While cfDNA from healthy donors showed rather low background of DNA methylation across the marker set, the lung cancer patient samples showed an overall higher level of the DNA methylation signal and a substantial fraction of the patients showed high level of DNA methylation across majority of the markers (FIG. 5A, FIG. 9). Eighty-three percent of patients have the DNA methylation signal higher than the 95^{th} percentile of the control group (FIG. 9). The distribution of the mean DNA methylation signal from all markers in the group of NSCLC patients (cases) is highly significantly different (p-value=1.6×10-8) from the group of healthy individuals (controls) (FIG. 5A). The median methylation per marker is about 29-fold higher in the cases than in the controls (FIG. 5A). The ROC analysis using the 47 controls and 18 cases revealed quite large area under the curve (AUC=0.956) with 95% confidence interval 0.906-1.0 (FIG. 5B). These findings clearly illustrate that the marker set, and the adopted detection technique are able to distinguish between the plasma from healthy individuals and the plasma from lung cancer cases with high sensitivity and specificity.

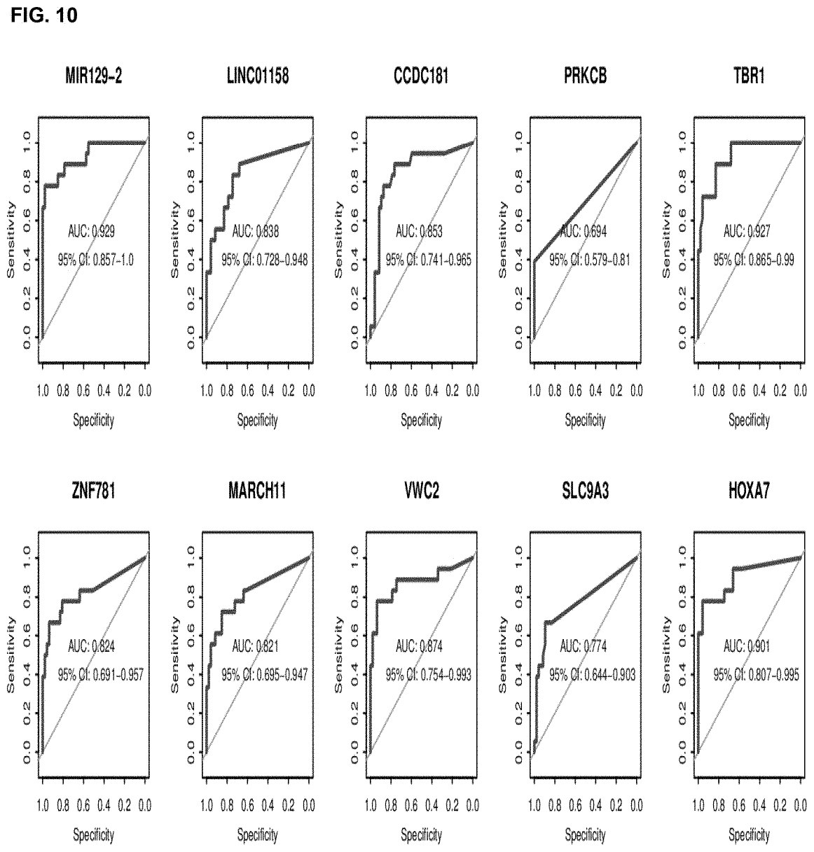

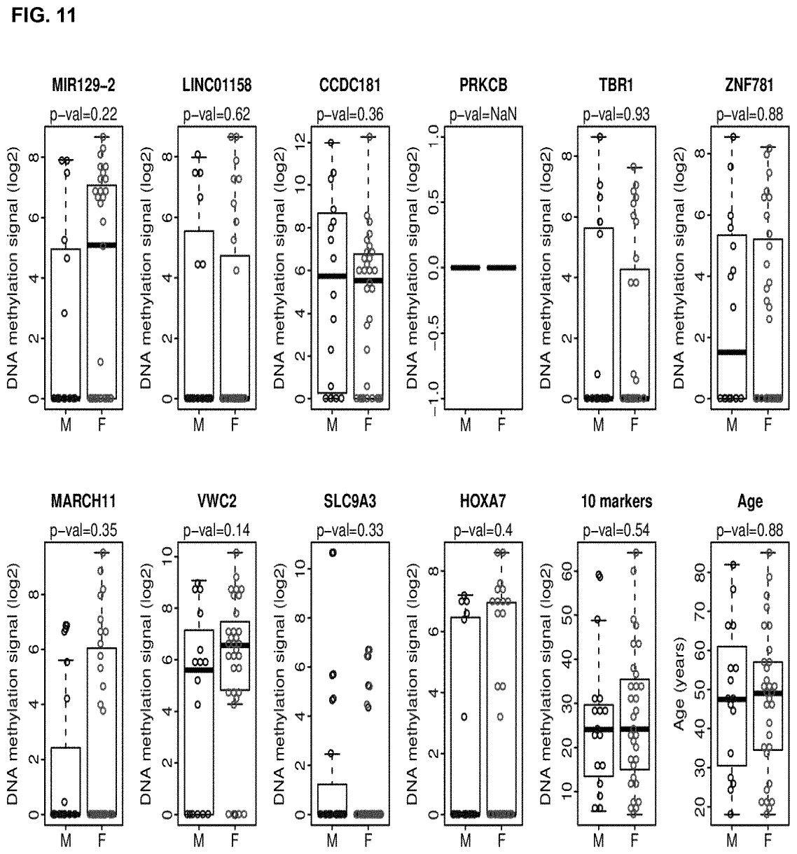

Next, each marker was evaluated separately using the same plasma sample sets as described above. The AUC for the individual markers ranged from 0.694 to 0.929 (FIG. 10), an optimal subset of five biomarkers was determined, which is less than the full marker set, and it indicates benefit of combination of multiple markers. No significant differences were revealed when comparing individual marker methylation between sexes in healthy controls (FIG. 11).

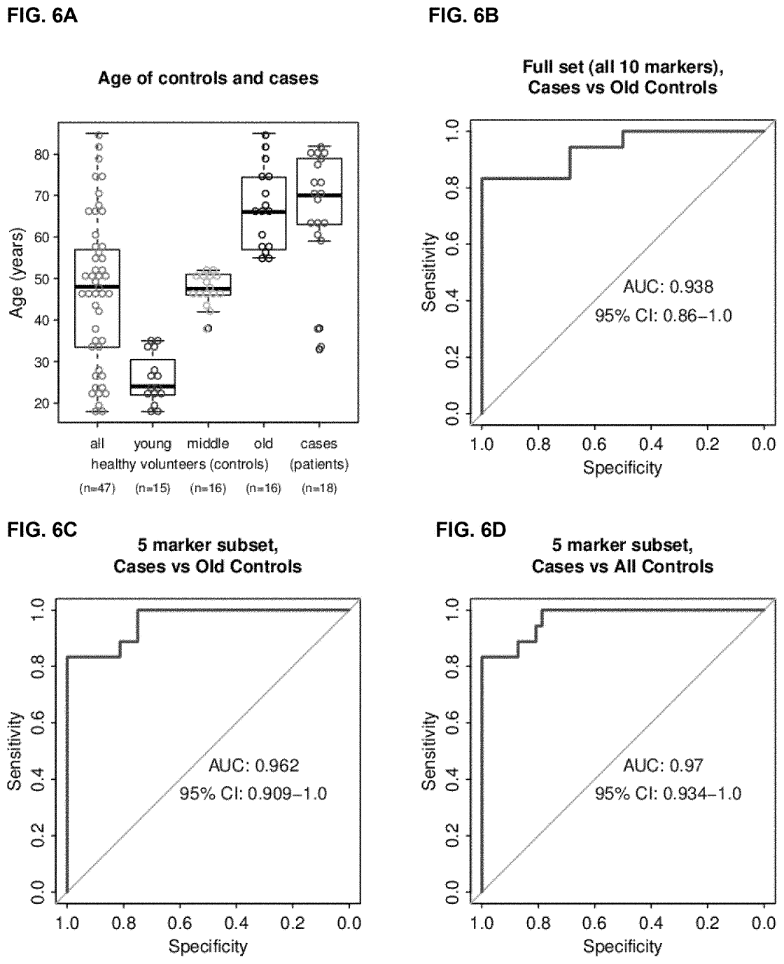

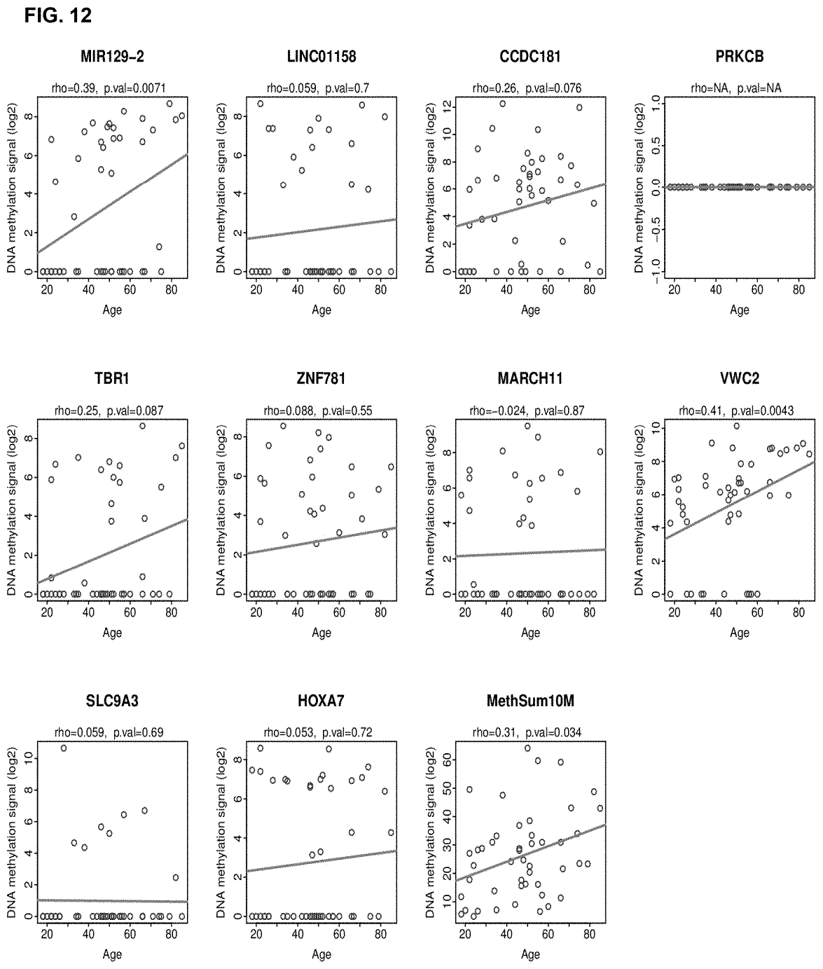

DNA methylation is known to change with age, next the relationship between DNA methylation levels of individual markers and age of healthy subjects was analyzed. As expected, some of the markers have increased in methylation with age (FIG. 12). On average the background DNA methylation signal per marker increased about 2.5 fold between healthy subject of ages 25 years and 75 years (FIG. 12); however, this is much lower difference than the 29 fold increase in cancer patients compared to healthy controls (FIG. 5A). However, the performance of the whole marker set using the control cohort separated by age into three sub-cohorts: young, middle and old age was analyzed (FIG. 6A). Even the oldest sub-cohort of controls which has an age distribution similar to the case cohort (FIG. 6A) was well separated by markers from the cancer patients (AUC=0.938, FIG. 6B). Nonetheless, this should be considered when using the markers for diagnostic purposes.

It was predicted that there would be a subset of markers within the full 10 marker set that will provide better separation between cases and controls. To address this prediction, an ROC analysis on all possible marker combinations using either the whole control cohort or the old control sub-cohort as healthy references was analyzed. The analysis determined a five marker subset that can separate cases from the old control sub-cohort with AUC=0.962 (0.909-1.0) (FIG. 6C); even better than the performance of the full 10 marker set using the whole control cohort as a reference (FIG. 5C, AUC=0.956) and this five marker subset can separate cases from the whole control cohort with even better AUC=0.97 (0.934-1.0) (FIG. 6D). Overall, although the background methylation of the markers increases with age, the markers are able to differentiate between cases and older control subjects with high sensitivity and specificity, the performance of the markers could be further improved by using only a specific marker subset. The analysis of the individual markers determined that there was an optimal subset of five biomarker.

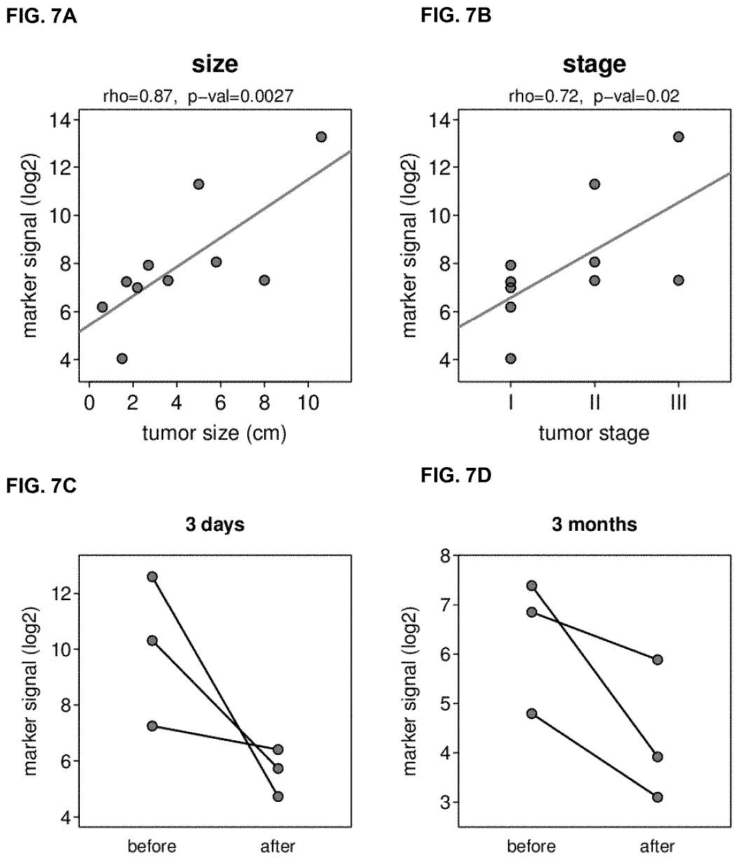

The signal from DNA methylation biomarkers in cfDNA samples from NSCLC patients depends on tumor size and disease stage and declines after tumor removal. Since the DNA methylation signal detected by the ten-marker set varied among individual patients (FIG. 5A), the correlation between the tumor size or disease stage and the signal of the full marker set was examined. A strong positive correlation between the tumor size and the marker signal (FIG. 7A) and also between the disease stage and the marker signal was observed (FIG. 7B). The fact that the strongest correlation of the marker signal (rho=0.87) was with the size of the tumor is consistent with quantitative nature of the assay; the larger the tumor the more ctDNA is sheds into bloodstream. To further test if the DNA methylation signal detected by the full marker set depends on the presence of a tumor in the body, we analyzed pairs of plasma samples from patients where samples were taken before the surgical resection of lung tumors and after either three days or three months post-surgery. Despite the limited number of sample pairs there was a clear trend towards substantially lower DNA methylation signal obtained from post-surgery samples; the level of decrease varied greatly from about two-fold to several hundred fold (FIGS. 7C-7D). The larger decreases in marker signal were observed in cases where the initial methylation signal was higher, i.e. the removed tumors were larger. This is again consistent with the quantitative nature of the assay. In summary, these observations indicate that the DNA methylation signal detected by the biomarkers depends on the presence of a tumor in the body and its size and that this noninvasive procedure could be used for monitoring cancer patients after intervention.

The data show highly significant differences in the level of DNA methylation of the marker loci between plasma cfDNA from lung cancer patients and control subjects. Furthermore, the signal from the markers depends on tumor size and decreases over time after definitive surgical resection of lung cancers, adding validity to the diagnostic value of the markers. The whole analytical procedure is relatively simple and could be performed using standard instrumentation. Since starting material (2 ml of plasma) could be obtained from a typical blood sample, the technique is minimally invasive. After cfDNA extraction and sodium bisulfite conversion, using commercially available kits, the technique involves two rounds of PCR; these can be performed on conventional PCR and qPCR instruments, respectively. The whole procedure could be accomplished by a single person within two days after the blood collection using conventional laboratory equipment and qPCR reagents. In summary, the technique is minimally invasive, simple, sensitive, fast and cost effective

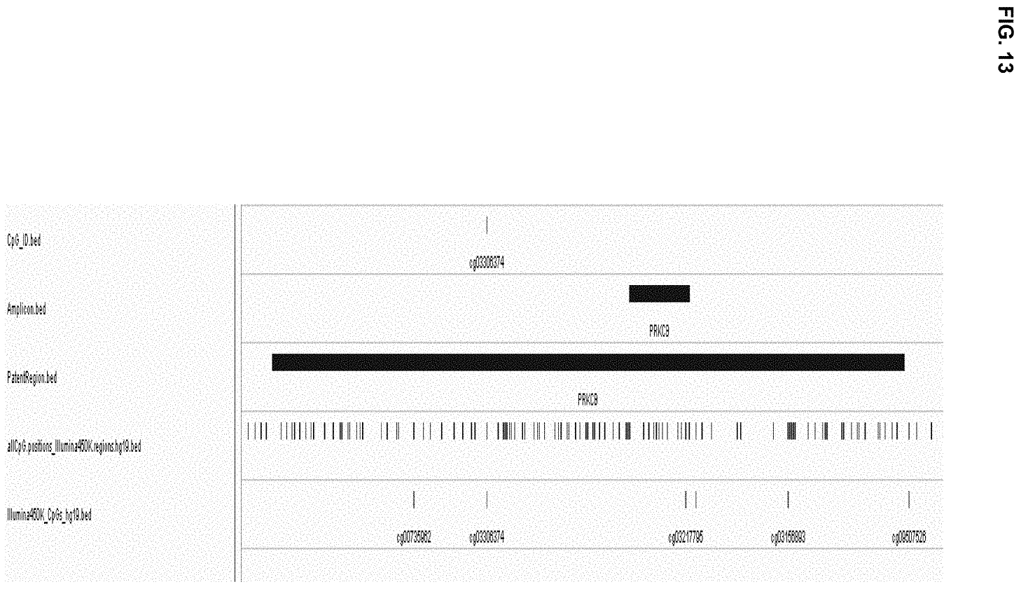

As disclosed herein, the inventors have found that there is an expanded region up to 250 bp in both directions from the upper or lower limit of an amplicon or the position of the marker CpG. In other words, discovery data involving Illumina CpG markers and the amplicons designed by the inventors are differentially methylated between cancer and normal samples, with the methylation region being found to be consistently differentially methylated through 500-750 bp. Thus, marker regions include both the position of the CpG from the discovery data as well as the qPCR amplicon region that are expanded 250 bp or more in both directions. Accordingly, region sizes typically will be in a range about 550-750 bp as seen in, for example, FIG. 13 and Table 4.

TABLE 4

| | | Patent. Region | CpG.ID_genomic. | Amplicon _genomic. | |
|---|---|---|---|---|---|
| Name | CpG.ID | (hg19) | position (hg19) | position (hg19) | Size |
| MIR129-2 | cg14416371 | chr11: 43602597- | chr11: 43602847- | chr11: 43602876- | 598 |
| (amplicon 70bp) | | 43603195 | 43602848 | 43602945 | |
| LINC01158 | cg08189989 | chr2: 105458914- | chr2: 105459164- | chr2: 105459225- | 646 |
| (amplicon 86bp) | | 105459560 | 105459165 | 105459310 | |
| CCDC181 | cg00100121 | chr1: 169396385- | chr1: 169396635- | chr1: 169396658- | 609 |
| (amplicon 87bp) | | 169396994 | 169396636 | 169396744 | |
| PRKCB | cg03306374 | chr16: 23847075- | chr16: 23847325- | chr16: 23847491- | 736 |
| (amplicon 71bp) | | 23847811 | 23847326 | 23847561 | |
| TBR1 | cg01419831 | chr2: 162283352- | chr2: 162283705- | chr2: 162283602- | 604 |
| (amplicon 73bp) | | 162283956 | 162283706 | 162283674 | |
| ZNF781 | cg25875213 | chr19: 38182805- | chr19: 38183055- | chr19: 38183080- | 602 |
| (amplicon 78bp | | 38183407 | 38183056 | 38183157 | |
| MARCH11 | cg00339556 | chr5: 16179798- | chr5: 16180048- | chr5: 16180057- | 597 |
| (amplicon 89bp) | | 16180395 | 16180049 | 16180145 | |
| VWC2 | cg01893212 | chr7: 49812797- | chr7: 49813088- | chr7: 49813047- | 569 |
| (amplicon 70bp) | | 49813366 | 49813089 | 49813116 | |
| SLC9A3 | cg14732324 | chr5: 528326- | chr5: 528621- | chr5: 528576- | 578 |
| (amplicon 79bp) | | 528904 | 528622 | 528654 | |
| HOXA7 | cg07302069 | chr7: 27196014- | chr7: 27196286- | chr7: 27196264- | 567 |
| (amplicon 68bp) | | 27196581 | 27196287 | 27196331 | |

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

1. A method for preparing a deoxyribonucleic acid (DNA) fraction from a subject useful for analysing genetic loci involved in DNA methylation, comprising:
a. extracting DNA from substantially cell-free sample of blood plasma or blood serum of the subject to obtain cell free DNA (cfDNA)
b. producing a fraction of the cfDNA extracted in (a) by:
i. treating cfDNA with sodium bisulfite (BS) to produce either a set of uracil modified cfDNA and a set of methylated cfDNA and;
ii. selectively amplifying only the methylated cfDNA of at least 2 methylation biomarker;
wherein the cfDNA fraction after (b) comprises a plurality of genetic loci of the cfDNA, and;
c. quantifying and analysing the methylation at a plurality of genetic loci of the cfDNA fraction produced in (b).

2. (canceled)

3. (canceled)

4. The method of claim 1, wherein amplifying the methylated cfDNA comprises use of a polymerase chain reaction (PCR).

5. The method of claim 1, wherein the quantifying or the analysing of the methylated cfDNA comprises use of quantitative PCR (qPCR).

6. (canceled)

7. The method of claim 1, wherein methylation biomarkers are selected from the group consisting of those with a genomic position of: chr11:43602597-43603195, chr2:105458914-10545960, chr1:169369385-16939694, chr16:23847075-23847811, chr2:162283352-162283956; chr19:38182805-38183407, chr5:16179798-16180395, chr7:49812797-49813366, chr5:528326-528904, and chr7:27196014-27196581.

8. (canceled)

9. The method of claim 7, wherein 5-10 biomarkers are selected for amplifying methylated DNA.

10. The method of claim 7, wherein 8-10 biomarkers are selected for amplifying methylated DNA.

11. A method of treating a plurality of cancers by administering anti-cancer therapeutics in a subject with cancer, the method comprising the steps of:
a. determining the subject's DNA methylation level by:
i. extracting DNA from substantially cell-free sample of blood plasma or blood serum of M subject to obtain cell free DNA (cfDNA)
ii. producing a fraction of the cfDNA extracted in (i) by:
1. treating cfDNA with sodium bisulfite (BS) to produce either a set of uracil modified cfDNA and a set of methylated cfDNA and;
2. selectively amplifying only the methylated cfDNA of at least 2 methylation biomarker;
wherein the DNA fraction after (ii) comprises a plurality of genetic loci of the cfDNA, and;
iii. quantifying and analysing the methylation at a plurality of genetic loci of the cfDNA produced in (ii).

12. (canceled)

13. (canceled)

14. The method of claim 11, wherein amplifying the methylated cfDNA comprises use of a polymerase chain reaction (PCR).

15. The method of claim 11, wherein the quantifying or the analysing of the methylated cfDNA comprises use of quantitative PCR (qPCR).

16. (canceled)

17. The method of claim 11, wherein methylation biomarkers are selected from the group consisting of those with a genomic position of: chr11:43602597-43603195, chr2:105458914-10545960, chr1:169369385-16939694, chr16:23847075-23847811, chr2:162283352-162283956; chr19:38182805-38183407, chr5:16179798-16180395, chr7:49812797-49813368, chr5:528326-528904, and chr7:27196014-27196581.

18. (canceled)

19. The method of claim 17, wherein 5-10 biomarkers are selected for amplifying methylated DNA.

20. The method of claim 17, wherein 8-10 biomarkers are selected for amplifying methylated DNA.

21. The method of claim 11, wherein said plurality of different cancer types comprises, urothelial bladder carcinoma (BLCA), breast invasive carcinoma (BRCA), colon adenocarcinoma (COAD), esophageal carcinoma (ESCA), head-neck squamous cell carcinoma (HNSC), lung adenocarcinoma (LUAD), lung squamous cell carcinoma (LUSC), pancreatic adenocarcinoma (PAAD), prostate adenocarcinoma (PRAD), and rectum adenocarcinoma (READ).

22. The method of claim 11, wherein the anti-cancer therapeutics consist of one or more of surgery, chemotherapy, radiation therapy, hormonal therapy, targeted therapy (including immunotherapy such as monoclonal antibody therapy) and synthetic lethality.

23. A method of detecting one or more cancers from a plurality of different cancer types in a subject, a method comprising:
a. extracting DNA from substantially cell-free sample of blood plasma or blood serum of the subject to obtain cell free DNA (cfDNA)
b. producing a fraction of the cfDNA extracted in (a) by:
i. treating cfDNA with sodium bisulfite (BS) to produce either a set of uracil modified cfDNA and a set of methylated cfDNA and;
ii. selectively amplifying only the methylated cfDNA of at least 2 methylation biomarker;
wherein the cfDNA fraction after (b) comprises a plurality of genetic loci of the cfDNA, and;
c. quantifying and analysing the methylation at a plurality of genetic loci of the cfDNA produced in (b).

24. (canceled)

25. (canceled)

26. The method of claim 23, wherein amplifying the methylated cfDNA comprises use of a polymerase chain reaction (PCR).

27. The method of claim 23, wherein the quantifying or the analysing of the methylated cfDNA comprises use of quantitative PCR (qPCR).

28. (canceled)

29. The method of claim 23, wherein methylation biomarkers are selected from the group consisting of those with a genomic position of: chr11:43602597-43603195, chr2:105458914-10545960, chr1:189369385-16939694, chr16:23847075-23847811, chr2:162283352-162283956; chr19:38182805-38183407, chr5:16179798-16180395, chr7:49812797-49813366, chr5:528326-528904, and chr7:27196014-27196581.

30. (canceled)

31. The method of claim 29, wherein 5-10 biomarkers are selected for amplifying methylated DNA.

32. (canceled)

33. The method of claim 23, wherein said plurality of different cancer types comprises, urothelial bladder carcinoma (BLCA), breast invasive carcinoma (BRCA), colon adenocarcinoma (COAD), esophageal carcinoma (ESCA), head-neck squamous cell carcinoma (HNSC), lung adenocarcinoma (LUAD), lung squamous cell carcinoma (LUSC), pancreatic adenocarcinoma (PAAD), prostate adenocarcinoma (PRAD), and rectum adenocarcinoma (READ).
